(19) United States
(12) Patent Application Publication
(10) Pub. No.: US 2005/0250101 A1
(43) Pub. Date: Nov. 10, 2005

(54) Method for identifying vehicle and oligonucleotide marker used therefor

(76) Inventors: Jun-mo Gil, Chungcheongbuk-do (KR); Young-hee Kim, Chungcheongbuk-do (KR); Han-oh Park, Yuseong-gu (KR); Sang-Joo Lee, Chungcheongbuk-do (KR)

Correspondence Address: NATH & ASSOCIATES, 1030 15th STREET, NW, 6TH FLOOR, WASHINGTON, DC, 20005, US

(21) Appl. No.: 10/531,271

(22) PCT Filed: Oct. 16, 2003
(86) PCT No.: PCT/KR03/02162

§ 371 Date: Jul. 13, 2005

(30) Foreign Application Priority Data
Oct. 16, 2002 (KR) 10-2002-0063057

(51) Int. Cl. C12Q 1/68

(52) U.S. Cl. 435/6

(57) ABSTRACT

The present invention relates to a method for identifying vehicle and oligonucleotide marker used therefor. More particularly, the present invention is directed to a method for identifying vehicle by using oligonucleotide to which phase transfer agent is bound, and oligonucleotide marker used therefor.

### TECHNICAL FIELD

The present invention relates to a method for identifying vehicle and oligonucleotide marker used therefor. More particularly, the present invention is directed to a method for identifying vehicle by using oligonucleotide to which phase transfer agent is bound, and oligonucleotide marker used therefor.

### BACKGROUND ART

Even a small amount of oligonucleotides can be amplified to a large amount of oligonucleotides which have same base sequence, by polymerase chain reaction (hereinafter, PCR). The sequence of the original oligonucleotides which exist in very small amount, can be determined by analyzing the base sequence of amplified oligonucleotides.

Therefore, a small amount of oligonucleotides can be used for tracing the original source or conveyance path of materials or products such as oils, paint products, foodstuffs, or explosive compositions. In addition, by adding the oligonucleotides to a high-priced work of art, its authenticity can be verified exactly.

Oligonucleotide, which have many phosphodiester bonds, have hydrophilic property because oligonucleotides are anionically charged by deprotonation at neutral pH.

Therefore, oligonucleotide is easily dissolved in aqueous solutions, but insoluble in organic solutions. This property brings about a problem when oligonucleotide is tried to be dissolved in organic solution.

Meanwhile, some methods which employ oligonucleotide as a marker of an object, have been described in the patents, such as WO87/06383, WO90/14441, WO91/17265, and WO94/04918.

The oligonucleotide which can be used as a marker is described in WO87/06383. However, verification methods by using DNA amplification or base sequencing are not mentioned therein. In addition, any method for dissolving hydrophilic DNA into organic solution is not described in this patent at all.

WO90/14441 describes that oils and other kinds of non-polar liquid can be tagged effectively by adding detergents to the taggant prior to the addition of the taggant to the non-polar liquids in order to dissolve hydrophilic oligonucleotide in oils. However, the specification do not disclose a coding and labeling function of DNA sequence although it describes that the invention can be used to verify the existence of the DNA by detecting the amplified product of the nucleic acid.

In WO91/17265, as it is in WO90/14441, it is described that the base sequence of oligonucleotides can be determined by amplification of the DNAs by using specific primer. It is also disclosed that oligonucleotides can be covalently linked to a substance or supporting materials in solid phase. However, in case that nucleotide is directly linked to paint or oil, it is required to break covalent bonds at the steps of extraction and recovery of oligonucleotides. In spite of the above, the process described in this patent has a problem for commercialization because the process may modify bases so that oligonucleotide cannot be amplified with the exact sequence.

WO94/04918 teaches an art using two or more particles containing two or more different markers, i.e., fluorescent, luminescent, phosphorescent or other kinds of labels capable of producing photometric signals. However, this method also does not consider the reactivity of hydroxyl groups of oligonucleotides or amino groups of bases. Therefore, still it has a problem that oligonucleotides with original base sequence cannot be obtained through PCR.

Moreover, the methods used in the above patents can be only applicable for paint or oil. This patent suggests a method of coding a vehicle with DNA sequence information and tracing or verifying of the vehicle by using the code.

For the purpose of overcoming the problems of these prior arts mentioned above, the applicant of the present invention have disclosed the oligonucleotides which have improved solubility in lipophillic solvents and a method of identifying objects using them in Korean patent application No. 2001-0037253. However, in the above application, oligonucleotide having particularly appropriate compatibility with vehicle painting materials or oligonucleotide which can be mixed easily with vehicle painting are not described.

Therefore, with an increase in traffic accidents caused by an increase in number of vehicles and traffic jam, the development of more effective and efficient method for tracing and identifying of hit-and-run vehicles, has been expected.

The object of the present invention is to provide an oligonucleotide marker for vehicle identifying.

Another object of the present invention is to provide a method for marking of vehicles by using an oligonucleotide marker, which comprises:

i) a step for the linkage formation between the oligonucleotide containing coding sequence region and phase transfer agent in organic solvent;

ii) a step for eliminating reactivity by adding a protective group to the oligonucleotide bound to the phase transfer agent;

iii) a step for adding the oligonucleotide of which reactivity was eliminated, to vehicle painting materials; and

iv) a step for applying the vehicle painting materials to vehicles.

Yet, another object of the present invention is to provide a method for identifying vehicles, which comprises:

i) a step for extracting the marker from materials collected from vehicles labeled with the marker in which the oligonucleotide containing coding sequence region bound to phase transfer agent and protected by protective groups;

ii) a step for removing the protective group bound to the oligonucleotide, from the extracted marker;

iii) a step for analyzing the base sequence of the oligonucleotide; and

iv) a step for searching vehicles labeled with the maker having analyzed sequence from the step iii)

### BRIEF DESCRIPTION OF THE DRAWINGS

The above objects and other advantages of the present invention will become more apparent by describing in detail a preferred embodiment thereof with reference to the attached drawings, in which:

FIG. 1 is a schematic diagram of the process of the present invention.

FIG. 2 illustrates the complex of phase transfer agent and oligonucleotide derivative of the present invention.

FIG. 3 is a photograph of electrophoresis of the product obtained from PCR of the oligonucleotides after being diluted by each concentration.

FIG. 4a is a photograph of electrophoresis of the product obtained from PCR of the oligonucleotides recovered from the paint coated on a vehicle, which contains oligonucleotide derivatives protected with acryloylchloride and phase transfer agent.

FIG. 4b is a photograph of the base sequences of the oligonucleotides of FIG. 4a.

FIG. 5a is a photograph of electrophoresis of the product obtained from PCR of the oligonucleotide recovered from the paint coated on a vehicle, which contains oligonucleotide derivatives protected with acetyl chloride and phase transfer agent.

FIG. 5b is a photograph of the base sequences of the oligonucleotides of FIG. 5a

FIG. 6a is a photograph of electrophoresis of the product obtained from PCR of the oligonucleotide recovered from the paint coated on a vehicle, which contains oligonucleotide (A)-phase transfer agent complex and oligonucleotide derivative (B)-phase transfer agent.

FIG. 6b is a photograph of electrophoresis which shows the results of base sequencing of complex A and B of FIG. 6a.

FIG. 7a is a photograph of electrophoresis of product obtained from PCR of the oligonucleotide recovered from the paint coated on an object, which contains three (3) kinds of oligonucleotide derivatives with different base sequence-phase transfer agent complex.

FIG. 7b is a photograph of electrophoresis which shows the result of sequencing of three different oligonucleotide derivatives-phase transfer agent complex of FIG. 7a.

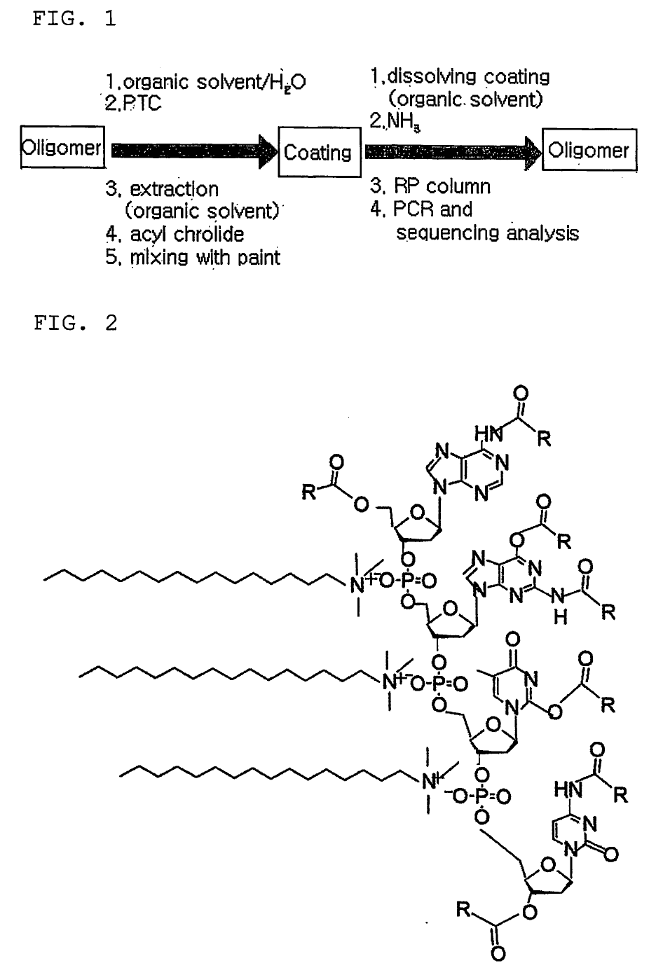

### DISCLOSURE OF INVENTION

The object of the present invention can be achieved by providing a vehicle identifying marker composed of oligonucleotide to which phase transfer agent is bound.

The oligonucleotide of the present invention is composed of coding sequence and PCR primer sequence positioned at both ends of the coding sequence.

The method for marking of vehicles by using an oligonucleotide marker of the present invention, is that the oligonucleotide is added to vehicle painting materials, such as vehicle painting dye, vehicle coating solution, lacquer and coating paint and, the vehicle painting materials are applied to vehicles.

The oligonucleotide of the present invention is preferably in the form of a derivatives of oligonucleotide protected with protective groups which block reactivity of the oligonucleotide.

The oligonucleotide of the present invention is composed of 10 to 50 base pairs coding sequence, and may be a combination of two or more kinds of oligonucleotides with different base sequences, and more preferably, three kinds of oligonucleotides with different base sequences.

The phase transfer agent of the present invention is quaternary ammonium compound or cationic surfactant, more preferably, is tetrabutyl ammonium hydroxide or hexadecyl trimethyl ammonium bromide.

The oligonucleotide derivative bound to phase transfer agent, may be prepared through the process which comprises 1) a step for the ion linkage formation between the oligonucleotide and phase transfer agent in organic solvent, 2) a step for eliminating reactivity by adding a protective group such as acyl halide to the oligonucleotide bound to the phase transfer agent.

The phase transfer agent in the form of quaternary ammonium compound or cationic surfactant can neutralize the negative charge of oligonucleotide by forming the ionic linkage with oligonucleotide. Therefore, oligoneucleotide neutralized by binding to the phase transfer agent can be dissolved in non-polar solvent such as organic solvent.

Lipo-soluble oligonucleotide can be dispersed homogeneously when it is mixed with lipophilic materials, such as oil paint. In the present invention, lipophilic materials containing oligonucleotide with extremely low concentration can be prepared.

The hydroxyl group at 5′ or 3′ of oligonucleotides bound to phase transfer agent from step i), reacts with the protecting group such as acyl halide in organic solvent, and thus is esterified. The amine group of oligoonucleotide reacts with protecting group to form amide linkage.

The acyl halide may be selected according to the type of organic solvents or the use of oligonucleotides. For example, in case of dissolving the oligonucleotide in paint, it is desirable to block the reactivity by using acyl halide(i.e. acetyl chloride) which has inactive substitute. In case of requiring chemical bond with component resin of paint, it is desirable to employ acyl halide(i.e. acryloylchoride) which have a reactivity inducing the chemical bond.

Moreover, the protecting group is selected from the group consisting of carbonyl compounds which form amide linkage with nitrogen and form ester linkage with oxygen, silanyl compounds which form N—Si bond and O—Si bond, sulfonyl compounds which form N—S bond and O—S bond, saturated carbohydrates, aromatic carbohydrates, unsaturated carbohydrates which form N—C bond and O—C bond can be broken by ammonia, saturated carbohydrates comprising hetero atoms or unsaturated carbohydrate comprising hetero atoms.

The oligonucleotide bound to phase transfer agent may react with the oily products When it is mixed with oily products. Therefore, the oligonucleotide thus reacted with oily product, cannot be recovered easily at the later recovery step.

Therefore, it is possible to block side reactions and insure chemical stability, by introducing acyl halide as a protecting group before oligonucleotide-phase transfer agent complex is added to oily product. Moreover, the loss of oligonucleotides occurred by chemical reactions with oily materials, can be minimized.

Another object of the present invention can be achieved by providing a method for marking vehicles by using an oligonucleotide marker, which comprises:

i) a step for the linkage formation between the oligonucleotide containing coding sequence region and phase transfer agent in organic solvent;

ii) a step for eliminating reactivity by adding a protective group to the oligonucleotide bound to the phase transfer agent;

iii) a step for adding the oligonucleotide of which reactivity was eliminated, to vehicle painting materials; and

iv) a step for applying the vehicle painting materials to vehicle.

Yet, another object of the present invention can be achieved by providing a method for identifying vehicles, which comprises:

i) a step for extracting the marker from materials collected from vehicles labeled with the marker in which the oligonucleotide containing coding sequence region bound to phase transfer agent and protected by protective groups;

ii) a step for removing the protective group bound to the oligonucleotide, from the extracted marker;

iii) a step for analyzing the base sequence of the oligonucleotide; and

iv) a step for searching vehicles labeled with the maker having analyzed sequence from the step iii)

### BEST MODE FOR CARRYING OUT THE INVENTION

Hereinafter, the present invention will be described in more detail.

The oligonucleotide of the present invention is composed of coding sequence and PCR primers positioned at both ends of the coding sequence.

It is desirable to use the oligonucleotides combined with protecting groups. The coding sequence region of oligonucleotides of the present invention, may be composed of 10 to 50 base pairs.

In the method of the present invention, it is possible to use a combination of two or more kinds of oligonucleotides with different base sequences, and more preferably, three kinds of oligonucleotides with different base sequences.

Moreover, the method of the present invention can further comprise a step for cloning of oligonucleotides to a vector after the amplification of the oligonucleotides.

Oligonucleotides bound to phase transfer agent of the present invention, which can be dissolved in organic solvent and of which reactivity is eliminated by protecting group, can be widely used as a marker for various kinds of oils, paints, foodstuffs, security systems or vehicles.

It is possible to trace hit-and-run vehicles by using oligonucleotide which have specific sequence of the present invention as a vehicle marker.

A fragment of the paint off from a hit-and-run vehicle coated with painting material containing oligonucleotide marker of the present invention, may be collected from the spot where the accident takes place. Then, the oligonucleotide is extracted from the fragment of the present invention. Next, the base sequence of oligonucleotide amplified by PCR can be analyzed.

In the above method, the sequence of oligonucleotide can function as a code(identifying marker) by base combination of A(adenine), C(cytosine), G(guanine), and T (thymine). For example, if an oligonucleotide is composed of 40 base pairs, each 15 base pair terminal region is set as a PCR primer binding region of known sequence and a 10 base pair central region is set as a code region.

Then, the code region can function as a code of which the number of cases is 4¹⁰ because it is made of combinations of the above four kinds of bases. Therefore, it is possible to label a very large number of objects with oligonucleotides which have different base sequence.

It is possible to identify the original object by comparison with the base code information through the method of the present invention and oligonucleotide marker database of the objectse.

Furthermore, when the number of object for identification is very large, it is possible to use a combination of three kinds of oligonucleotides with different base sequences. 4¹⁰ kinds of combincation of base pairs can be made in case that each coding region of three oligonucleotide is composed of 10 base pairs. Consequently, 4¹⁰×4¹⁰×4¹⁰ kinds of combination can be made by combining three kinds of oligonucleotide coding sequence. These great number of codes can cover to mark great number of vehicles.

When the above three kinds of oligonucleotide are designed, it is desirable to let each of terminal region composed of 15 base pairs have a unique sequence. It is also required that the sequence of terminal region does not have the same sequence with central code sequence region.

When the oligonucleotides, which has a code sequence region made from a combination of base sequence as an identifying marker, are used, the methods of labeling and code sequence recognition are as followings.

In the present invention, oligonucleotide base sequence is designed under the consideration of amplification by PCR. More concretely, 10 base pair coding region is synthesized from combinations of 10 base pairs (4¹⁰ kinds of identification markers can be allowed.), then, on its both ends, 15 base pair oligonucleotides of known sequence are positioned. Consequently, 40 base pair oligonucleotides were synthesized. 15 base pair regions of known sequence at both ends function as templates for forwarding and reversing primers, respectively, for PCR. If needed, code sequence region (10 base pairs) can be extended to more than 10 base pairs to increase the number of cases to allow more objects to be identified.

The oligonucleotides are designed as following:

1) Sequence Design of Code Sequence Region

Smith algorithm was used for basic design. First, a unique code sequence region was randomly produced for each oligonucleotide. After local alignment of the code sequences, oligonucleotides of similar sequences or cross hybridization were excluded (Smith, T. F. and M. S. Waterman, 1981, Identification of common molecular subsequences. J. Mol. Biol. 147, 195-197).

This process was performed by dynamic programming method with mismatch penalty of 3, match score of 10, and gap penalty of 3 for similarity measurement parameters. Only sequences with local alignment value of less than 75 were selected. Code sequence region of Oligonucleotide sequences were created through the above process, and formed into a database.

2) Sequence Design of Both Ends

15 base pair oligonucleotide sequences were randomly produced described as above. Among them, only those of melting point (Tm) of 50 to 55° C. were selected. Tm was obtained by nearest-neighbor method (Breslauer, K. J., Frank, R., Blocker, H., and Marky, L. A., 1986, Predicting DNA duplex stability from the base sequence. Proc. Natl. Acad. Sci. USA, 83, 3746-3750). This method is known to be more accurate than Guanine-Cytosine content value method.

The oligonucleotides can cause errors due to cross hybridization in the course of PCR. To solve this problem, only oligomers with local alignment value of less than 50 were selected after local alignment of forward and reverse primers. In addition, only oligomers with the sum of local alignment value of forward primer, code region, and reverse primer of less than 100 were finally selected. In this case, mismatch penalty, match score, and gap penalty were set 5, 10, and 5, respectively.

Oligonucleotides designed as above are synthesized by automatic oligonucleotide synthesizer and then purified. Next, aqueous solution of oligonucleotide and phase transfer agent are mixed in organic solvent (toluene or ethyl ether). After the mixture is phase-separated, only organic layer is selected. This procedure is repeated until oligonucleotide in aqueous layer is invisible by UV.

Oligonucleotides bound to phase transfer agent produced by this procedure form homogeneous phase against organic solvent, so it is possible to be dispersed in organic solvent at a extremely low concentration. Therefore, water-soluble oligonucleotides become lipo-soluble.

Oligonucleotides which are bound to phase transfer agent are used for lipophillic materials, for example, vehicle paint, lacquer, traffic lane paint, petroleum, paint diluent, explosives, natural oils, construction paint, organic solvent, adhesives, oily dyes, meat, and marine products.

However, amino groups or oxygen of base or hydroxyl groups of sugar, which are bound to phase transfer agent, according to the kind of lipophilic material, have reactivity with lipophilic material. Therefore, if the said oligonucleotides are mixed with lipophilic material directly, those may chemically react with the lipophilic material so that the said oligonucleotides could not be recovered effectively at later recovery step.

Therefore, before the said oligonucleotides bound to phase transfer agent are added to lipophilic material, it is desirable to block their reactive functional groups by introducing protecting groups using materials like acyl halide. Acyl halide used herein, are acetyl chloride which have non-reactive derivatives, or acryloyl chloride which have derivatives being able to make strong chemical bond with resin constituting paint. As a result, all the original oligonucleotides could be recovered as the following example.

The method for the recovery of oligonucleotides which are bound to phase transfer agent from the mixture of oligonucleotides and lipophilic material is as the following. For example, the method to extract oligonucleotides from a fragment of paint mixed with oligonucleotide derivatives which are bound to phase transfer agent is, first, to dissolve oligonucleotides from the paint fragment. The fragment is treated with organic solvent and dissolved oligonucleotides are extracted. Second, a trace of oligonucleotide is obtained. Then, to remove the protecting groups of oligonucleotide derivatives, the extracted oligonucleotide derivative is treated with ammonia. As a result, oligonucleotide is reduced to original phosphodiester structure.

The method to trace and identify vehicles from the extracted oligonucleotide is as the following. Oligonucleotides of which protecting groups are removed by treating with ammonia are amplified by PCR. Here, among 40 base pairs, 15 base pair regions have known sequences. Therefore, with the primers corresponding to these base sequenceS as forward and reverse primers, the oligonucleotide is amplified by PCR. Then, by analyzing the base sequence of the amplified product, it is possible to trace and identify vehicle labeled with a marker corresponding to the analyzed sequence. Through this procedure, the identity of object is verified.

Hereinafter, the present invention will be described in greater detail with reference to the following examples. The examples are given only for illustration of the present invention and not to be limiting the present invention.

### EXAMPLE 1

### Determination of the Concentration of Oligonucleotide

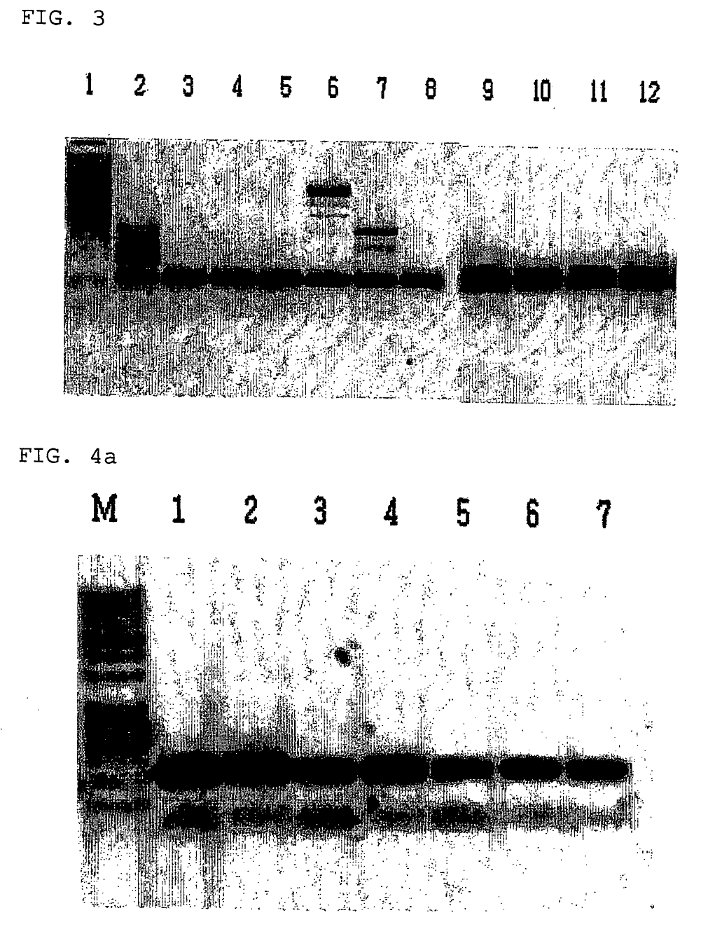

To determine the concentration of oligonucleotide which will be used to paint, 40 base pair oligonucleotide (40 mer) was synthesized. Then, it was serially diluted by tenfold from 10 pmole/ul to 1 ztmole/ul and was amplified by PCR. FIG. 3 represents the result of the agarose gel electrophoresis of the amplified product. As represented in FIG. 3, oligonucleotide could be amplified by PCR at even 1 ztmole.

In determining the concentration of oligonucleotide which is added to paint, the loss of oligonucleotide during the extraction from paint and the amount of oligonucleotide which is added to paint were considered. The concentration of oligonucleotide which is added to paint was determined as 100 atmole and used for the following experiment.

In FIG. 3, each lane represents the concentration of oligonucleotide. Lane 1 represents 10 pmole, lane 2; 1 pmole, lane 3; 100 ftmole, lane 4; 10 ftmole, lane 5; 1 ftmole, lane 6; 100 atmole, lane 7; 10 atmole, lane 8; 1 atmole, lane 9; 1 atmole, lane 10; 100 ztmole, lane 11; 10 ztmole, lane 12; 1 ztmole.

### EXAMPLE 2

### Testing Solubility of Oligonucleotide by Phase Transfer Agent for Organic Solvent

The designed oligonucleotides were synthesized by automatic oligonucleotide synthesizer and then purified. Next, aqueous solution of oligonucleotide and phase transfer agent were mixed in organic solvent (toluene or ethyl ether). After the mixture was phase-separated, only organic layer was retained. This procedure was repeated until oligonucleotide in aqueous layer was invisible by UV. With tetrabutyl ammonium hydroxide and hexadecyl trimethyl ammonium bromide as phase transfer agent, UV absorption was measured. From the results, the following was confirmed. In case phase transfer agent was not added, all the oligonucleotides were dissolved in aqueous layer (5 ml), but as a result of the addition of phase transfer agent, oligonucleotides were extracted from organic layer (toluene or ethyl ether 5 ml).

After organic solvent layer was extracted more than 5 times (5 ml each), aqueous layer was tested with UV. The results showed that most oligonucleotide dissolved in the organic layer and almost no oligonucleotide remained in the aqueous layer. Moreover, the Maldi-Tof mass spectrum for oligonucleotides bound to phase transfer agent, which were dissolved in the organic layer, showed that those oligonucleotide were same with the original oligonucleotide.

### EXAMPLE 3

### Experiment of Protecting Reaction to Introduce Protecting Groups into Oligonucleotide

Before the 40 base pair oligonucleotides, which is bound to phase transfer agent and dissolved in organic layer, were added to paint, experiment of protecting reaction to protect oligonucleotide was performed. The organic layer which contain oligonucleotides bound to phase transfer agent was reacted with excess acryloyl chloride. The salt from above reaction was extracted with aqueous solution and removed. Then, organic layer was analyzed by Maldi-Tof mass spectrometer. The result showed that, on average, 1.8 reactive groups were replaced by acryloyl groups in each base. Since acryloyl group include double bond and ketone, it can form covalent bonds by addition reaction with resin component of paint.

### EXAMPLE 4

### Experiment of Coating with the Mixture of Vehicle Coating Lacquer and Oligonucleotide Derivative Bound to Phase Transfer Agent and the Recovery of the Oligonucleotide

Oligonucleotide derivatives bound to phase transfer agent, which were composed of 40 base pairs (40 mer), were mixed with vehicle coating lacquer, then an experiment of coating and recovery was performed. After oligonucleotides bound to phase transfer agent were mixed with vehicle coating lacquer, a glass surface was coated with the mixture. Then, after the coated lacquer was dried in the oven of over 80° C. for more than 12 hours, it was cooled down and washed with water and detergent for many times. After that, to recover oligonucleotides from lacquer, the following procedure was performed. The remained fragment of lacquer was dissolved with acetonitrile and dimethylformamide (DMF), then, to remove protecting groups attached to amino groups of bases or hydroxyl groups of sugars of oligonucleotides, the reaction mixture was treated with ammonia for more than 12 hours at 80° C. Then, after lacquer component was extracted with ethyl ether, only aqueous layer was retained. Next, by passing through a short reverse-phase column, oligonucleotides were recovered in their original form. After recovered oligonucleotide was amplified by PCR, base sequencing was performed using the amplified product.

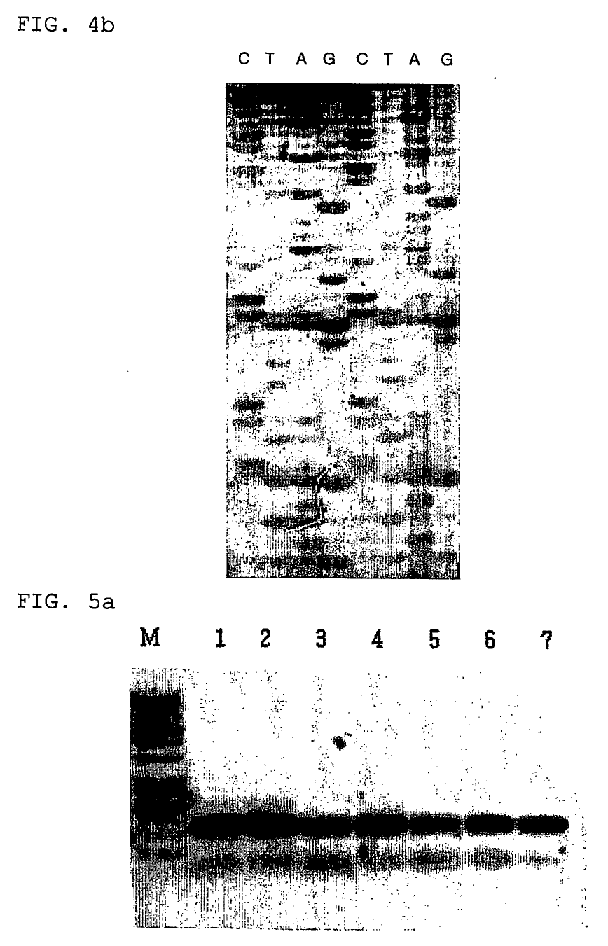

The procedure of PCR and base sequencing of the oligonucleotides was as the following. The sequence of the oligonucleotide composed of 40 base pairs (40 mer) was 5′-agc att ttg tgg ggc gtg ata gcc tcc ttg gcc gca aag a-3′, and in PCR, forward primer sequence was 5′-agc att ttg tgg ggc-3′ (15 mer), reverse primer sequence was 5′-cc ttg gcc gca aag acc acc acc tcg cgg (29 mer)-3′. To increase the efficiency of PCR, reverse primer was set as 29 base pairs (29 mer), not 15 base pairs (15 mer). To analyze base sequence of oligonucleotides which had been amplified by PCR, the amplified oligonucleotides were purified with DNA PrepMate II (DNA PrepMate II, product of Bioneer Corporation), and then, their base sequence was analyzed by direct base sequencing on 10% polyacrylamide gel. FIG. 4a and FIG. 4b present the results. FIG. 4a represents the results of agarose gel electrophoresis of the products obtained by the above procedure. From FIG. 4a, it is confirmed that oligonucleotide is recovered normally in the present invention. FIG. 4b represents the result of base sequencing of the amplified product of FIG. 4a. FIG. 4b shows that the sequence of original oligonucleotides and the sequence of amplified oligonucleotides coincided. Particularly, excluding the forward and reverse primer binding regions, the base sequence of code sequence region composed of 10 base pairs (gtg ata gcc t) coincided. Therefore, it was verified that forward primer and reverse primer recovered normally in the present invention. Therefore, it was verified that oligonucleotide could function as a marker by making each marker have different code sequence.

However, in case that the amplified product by PCR was base sequenced as it was, due to impurities, the photograph was not clear and a base very next to sequencing primer binding region was not analyzed. Then, base sequencing was not done for amplified product itself, but done after it is cloned into vector.

original base sequence: 5′-agc att ttg tgg ggc gtg ata gcc tcc ttg gcc gca aag a-3′

resulting base sequence: 5′-g ata gcc tcc ttg gcc gca aag acc acc acc-3′

In FIG. 4a, M represents a size marker, lane 1 to lane 7 represent the results of experiment with 100 atmole/ul oligonucleotides. In FIG. 4b, left four lanes represent the results of base sequencing of oligonucleotides recovered according to the present invention, right fours lane represent the results of base sequencing of oligonucleotides which had not been mixed with vehicle coating lacquer as a control group.

### EXAMPLE 5

### Experiment of Coating with the Mixture of Vehicle Coating Lacquer and Acetyl-Displaced Oligonucleotide Derivative, which is Bound to Phase Transfer Agent and the Recovery of Oligonucleotides

With the exception of using acetyl chloride instead of acryloyl chloride, the same procedure as example 3 was carried out. Then, oligonucleotide derivatives bound to phase transfer agent, which are composed of 40 base pairs (40 mer) were amplified by PCR and were base sequenced by the same procedure as example 4. Then, to analyze the base sequence of oligonucleotides amplified by PCR, they were purified with DNA PrepMate II (product of Bioneer Corporation). Then, purified oligonucleotides were cloned into T-vector and their base sequence was analyzed with 10% polyacrylamide gel using a primer which is complementary to T7 promoter of T-vector.

FIG. 5a and FIG. 5b represent the results. FIG. 5a represents the oligoneucleotides amplified by PCR, which is extracted from the mixture with vehicle coating lacquer after being coated to the vehcle.

From FIG. 5a, it is confirmed that oligonucleotide is recovered normally in the present invention. FIG. 5b shows that the sequence of original oligonucleotides and the sequence of amplified oligonucleotides coincided.

original base sequence : 5′-agc att ttg tgg ggc gtg ata gcc tcc ttg gcc gca aag a-3′

resulted base sequence: 5′-ggt ggt ctt tgc ggc caa gga ggc tat cac gcc cca caa aat gct-3′ (reverse cloned)

analyzed base sequence: 5′-agc att ttg tgg ggc gtg ata gcc tcc ttg gcc gca aag acc acc-3′

In FIG. 5a, M represents a size marker, lane 1 to lane 7 represent the results of the agarose gel electrophoresis for the amplified products, which were recovered from the coat made with coating composition composed of vehicle coating lacquer and oligonucleotide bound to phase transfer agent. FIG. 5b represents the results of base sequencing of the products of FIG. 5a, which show that the base sequence of recovered oligonucleotides had exactly the original base sequence.

### EXAMPLE 6

### Experiment of Painting with Paint Composition Including Oligonucleotide (A) which is Bound to Phase Transfer Agent or Oligonucleotide Derivative (B) which is Bound to Phase Transfer Agent and Displaced by a Protecting Group, and Recovery of Oligonucleotide

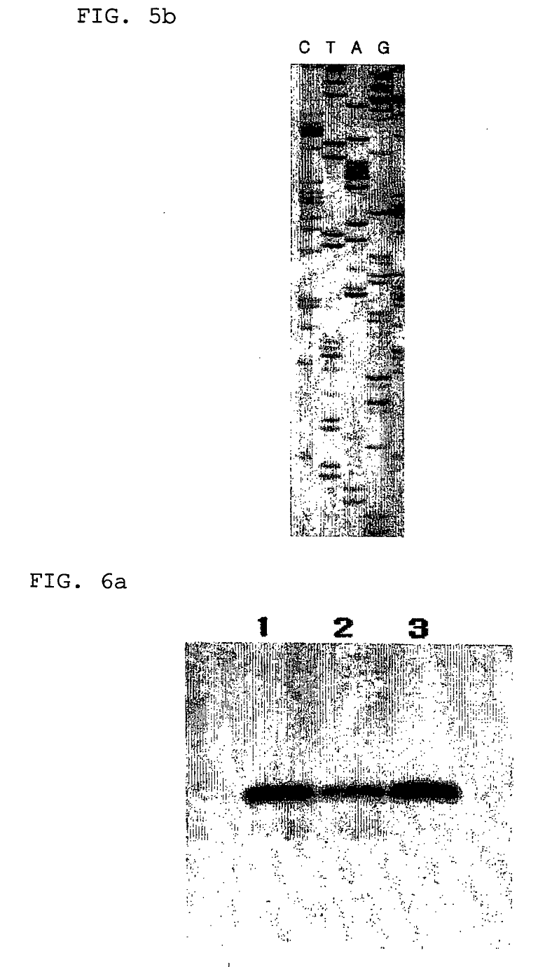

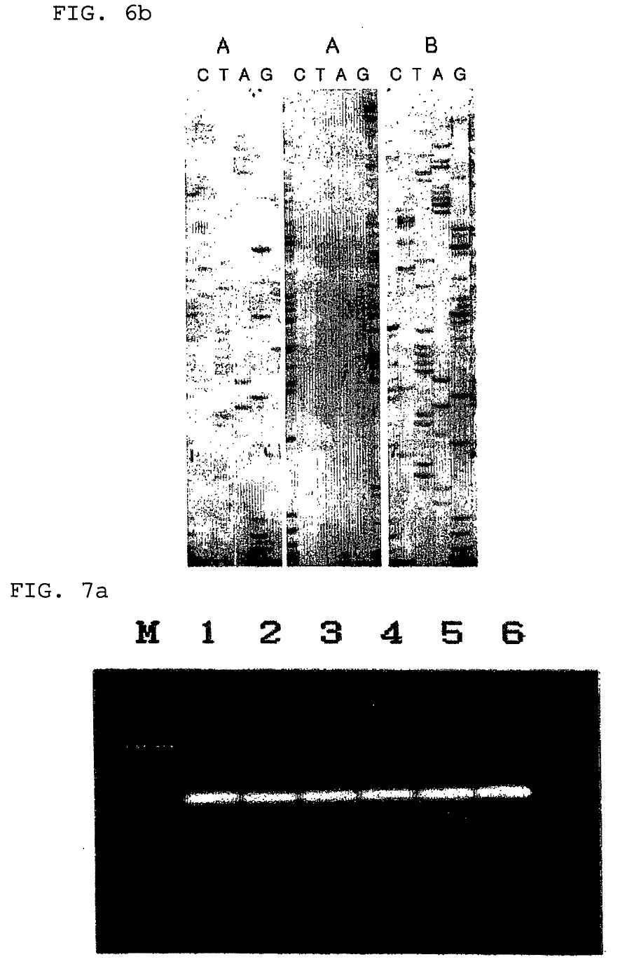

The oligonucleotides (A) which were bound to phase transfer agent made by example 2, oligonucleotide derivatives (B) which were bound to phase transfer agent and displaced by acryloyl group made by example 3, was mixed with the various type of paint. Then, they were dried and recovered by the same procedure as example 4. The products were amplified by PCR and base sequences were analyzed. FIG. 6a and FIG. 6b show the results. In FIG. 6a, lane 1 represents the result for binding complex A and urethane paint, lane 2 for A and vehicle coating paint, lane 3 for B and vehicle coating paint. Lane 2 shows that A was not recovered well. Then, base sequence was analyzed and the result is as the following.

expected base sequence : 5′-agc att ttg tgg ggc tgc ctg gcg ccc ttg gcc gca aag acc acc acc tcg cgg-3′

resulted base sequence of lane 1(A): 5′-agc att ttg tgg ggc tgc ctg gcg ccc ttg gcc gca aag acc acc acc tcg c-3′

resulted base sequence of lane 3(B): 5′-agc att ttg tgg ggc tgc ctg gcg gcc cac aaa atc gt-3′

The agarose gel electrophoresis was carried out to purify the amplified product of binding complex A by PCR, which was recovered from the mixture with vehicle coating paint. The results showed that the band was not formed well in the gel, and was not cloned into T-vector. In the present example, the sequence of forward primer for PCR was agc att ttg tgg ggc. The next 10 sequence (tgc ctg gcg c) was the sequence which functioned as a marker and it was confirmed that the base sequence coincided exactly. The sequence of reverse primer was 5′-cc ttg gcc gca aag acc acc acc tcg cgg-3′ (29 mer). For binding complex A, the result was different according to the type of paint. When mixed with urethane paint it was well recovered, so its base sequence was analyzed well. However, when mixed with vehicle coating paint, its base sequence analysis was failed because it was not cloned as it should be. These results showed that bases of the oligonucleotides reacted with vehicle coating paint directly and it resulted in poor recovery since protecting reaction for amino groups or oxygen atoms was not carried out.

In FIG. 6b, left four lane show the sequences of oligonucleotides of lane 1 of FIG. 6a, central four lanes show the sequences of oligonucleotide of lane 2 of FIG. 6a, right four lanes show the sequences of oligonucleotide of lane 3 of FIG. 6a.

### EXAMPLE 7

### Experiment Using a Combination Composed of Three (3) Kinds of Oligonucleotides of which Base Sequences are Different each other

In the present example, a combination of 3 groups of oligonucleotides (40 mer) of which base sequences are different each other was used. Each oligonucleotide herein used was made with different base sequence in order to be used as different primers. The central coding sequence region was designed not to overlap with terminal primer binding region. The sequences designed as above were as the followings.

Oligo sequence 1: ctg atg ggc cgc aac ctt cag tac att ttg ggc gca cca t

Oligo sequence 2: tca ttc ccc gac cgg agc agt cga tgg cgt ttc acc ggg t

Oligo sequence 3: cgc gcg gtg ttg aat tca tgg cca gtg gaa cgc ttt ccg c

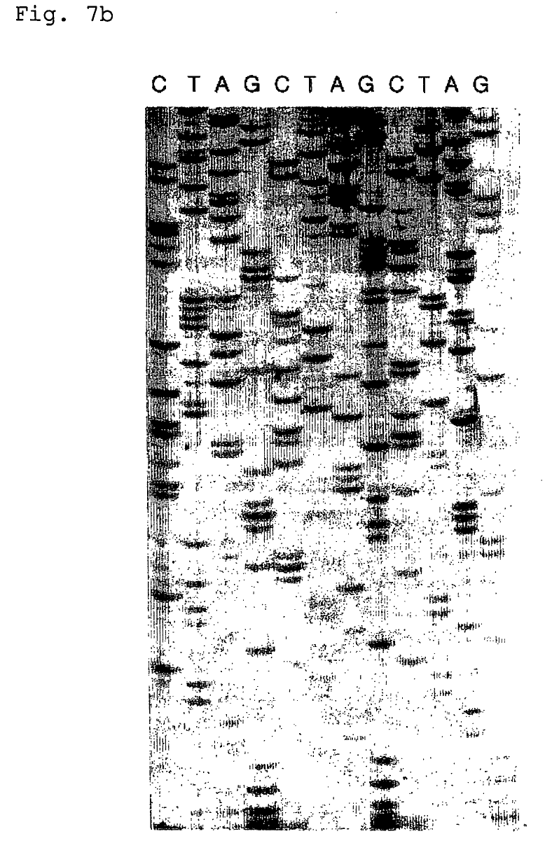

After the procedures of example 2 and example 3 were carried out, three (3) kinds of oligonucleotides which were bound to phase transfer agent made by example 4 were mixed with vehicle coating lacquer. Using the mixture, coating was carried out and dried, then, oligonucleotides were recovered from coated lacquer again. The recovered oligonucleotides were amplified by PCR. FIG. 7a represents the results. Each of primers used herein had different sequence and corresponded to each oligonucleotide. The primer's sequences used herein were as the followings.

primer 1 (forward: ctg atg ggc cgc aac, reverse: atg gtg cgc cca aaa)

primer 2 (forward: tca ttc ccc gac cgg, reverse: acc cgg tga aac gcc)

primer 3 (forward: cgc gcg gtg ttg aat, reverse: gcg gaa agc gtt cca)

Subsequently, the base sequence analysis was carried out. FIG. 7b represents the results.

In FIG. 7a, M represents a size marker, lane 1 and lane 2 represent the results of PCR amplification using primer 1, lane 3 and lane 4 represent the results of PCR amplification using primer 2, lane 5 and lane 6 represent the results of PCR amplification using primer 3.

In FIG. 7b, left fbur lanes represent oligonucleotide 1, central four lanes oligonucleotide 2, right four lanes oligonucleotide 3. The results of sequence analysis show that the sequence coincided with the original sequence.

Therefore, using a combination of three (3) groups of oligonucleotides of which base sequences are different each other, the original sequence could be identified. So, in real applications, combinations of two or more groups of oligonucleotides of which base sequences are different each other can be used. Since the number of cases increases by the combination of oligonucleotides, it can be used effectively when there are many objects to be labeled.

### INDUSTRIAL APPLICABILITY

The present invention provides a vehicle identifying marker and its application as a vehicle identification method utilizing oligonucleotides-phase transfer agent complex having code sequence region.

According to the present invention, when oligonucleotide derivatives bound to phase transfer agent, which are soluble in organic solvent, are added to various lipophilic materials, by extracting and analyzing the oligonucleotides, which were recovered from collected materials, it is possible to trace and identify the original object later. That is, if a vehicle is painted with a paint containing the said oligonucleotide derivatives bound to phase transfer agent, by identifying the code sequence from a small amount of paint, it is useful to trace the original vehicle. It can be also used for various similar purposes.

Since, by the present invention, it is possible to trace and identify hit-and-run vehicles from a fragment of paint after a traffic accident, the present invention is very useful to prevent hit-and-run accident and helpful to get conclusive evidence for the identification of hit-and-run vehicles.

While the present invention has been particularly shown and described with reference to a particular examples thereof, it will be understood by those skilled in the art that various changes and in form and details may be conceived there from without departing from the spirit and the scope of the present invention as defined by the appended claims.

1. A vehicle identifying marker composed of oligonucleotide to which phase transfer agent is bound.

2. The vehicle identifying marker according to claim 1, characterized in that said marker is added to materials selected from the group consisting of vehicle painting dye, vehicle coating solution, lacquer and coating paint.

3. The vehicle identifying marker according to claim 1, characterized in that said oligonucleotide is composed of coding sequence and PCR primer sequence positioned at both ends of the coding sequence.

4. The vehicle identifying marker according to claim 3, characterized in that said coding sequence is composed of 10 to 50 base pairs.

5. The vehicle identifying marker according to claim 1, characterized in that said oligonucleotide is a combination of two or more kinds of oligonucleotides with different base sequences.

6. The vehicle identifying marker according to claim 5, characterized in that said oligonucleotide is a combination of three kinds of oligonucleotides with different base sequences.

7. The vehicle identifying marker according to claim 1, characterized in that said oligonucleotide is further combined with protective groups which blocks reactivity.

8. A method for marking of vehicles by using an oligonucleotide marker, which comprises:
i) a step for the linkage formation between the oligonucleotide containing coding sequence region and phase transfer agent in organic solvent;
ii) a step for eliminating reactivity by adding a protective group to the oligonucleotide bound to the phase transfer agent;
iii) a step for adding the said oligonucleotide of which reactivity was eliminated, to vehicle painting materials; and
iv) a step for applying the said vehicle painting materials to vehicles.

9. A method for identifying vehicles, which comprises:
i) a step for extracting the said marker from materials collected from vehicles labeled with the marker in which the oligonucleotide containing coding sequence region bound to PHASE TRANSFER AGENT and protected by protective groups;
ii) a step for removing said protective group bound to said oligonucleotide, from said extracted marker;
iii) a step for analyzing the base sequence of said oligonucleotide ; and
iv) a step for searching vehicles labeled with said marker having analyzed sequence from the step iii)

10. The method according to claim 9, characterized in further comprising an amplification step of the oligonucleotide of said marker by PCR before sequence analysis step iii).

11. The method according to claim 10, characterized in further comprising a cloning step of the oligonucleotide to a vector after the amplification step of said oligonucleotide.

12. The method according to claim 9, characterized in that said oligonucleotide is a combination of two or more kinds of oligonucleotides with different base sequences.

13. The method according to claim 12, characterized in that said oligonucleotide is a combination of three kinds of oligonucleotides with different base sequences.
